(19) United States
(12) Patent Application Publication
(10) Pub. No.: US 2014/0330191 A1
(43) Pub. Date: Nov. 6, 2014

(54) ANKLE SUPPORTIVE SOCK

(71) Applicants: Pierre Charles, Haverton, PA (US); Chris Wright, Havertown, PA (US)

(72) Inventors: Pierre Charles, Haverton, PA (US); Chris Wright, Havertown, PA (US)

(21) Appl. No.: 13/875,554

(22) Filed: May 2, 2013

(51) Int. Cl. A61F 5/01

(52) U.S. Cl. CPC A61F 5/0127
USPC 602/27

(57) ABSTRACT

A pair of ankle supportive socks where each sock comprises a tubular body, where the tubular body includes a foot portion, a rise and an opening; an ankle brace attached to a backside of the tubular body; a pair of wings extending from the ankle brace, where the pair of wings wrap around the tubular body; a fastening means on the pair of wings, where the fastening means secures the wings to each other around the tubular body. The ankle supportive sock may include a padded toe and padded heel to provide additional comfort and support during wear.

### BACKGROUND OF THE INVENTION

1. Field of the Invention

The present invention relates to a pair of socks that provide additional ankle support for a wearer, where the ankle support may be adjusted as needed during use.

2. Description of Related Art

Ankle stress and injuries are common occurrences among athletes. Due to the high usage, especially for quick maneuvering, pivoting and running, the ankle is highly susceptible to damage and harm. Over time the cartilage, muscles and tendons that make up the joint weaken which may cause the likelihood of injury to increase. For this reason many people are required to wear a brace that provides additional strength and stability to the underlying joint. Also, for rehabilitative reasons, ankle braces are often prescribed by a doctor after the person injures themselves to allow the foot to heal without undue stress.

Most ankle braces are a tight fitting sleeve that fits around the arch, heel and ankle of the foot. Some braces may be more elaborately designed with straps, lacing and even reinforcement bars which maintain the ankle in a static position. Many braces are made to be worn while playing a sport which is convenient and optimal for the athlete. But a problem with these athletic braces is that they often become smelly and uncomfortable over time.

Therefore, it would be beneficial in the art to provide a comfortable ankle brace to support a wearer's ankle. It would also be desirable in the art to provide an ankle brace that is adjustable yet stable to reinforce the wearer's ankle during athletic activities.

### SUMMARY OF THE INVENTION

In view of the foregoing disadvantages inherent in the prior art, the general purpose of the present invention is to provide a sock with an ankle brace, configured to include all of the advantages of the prior art, and to overcome the drawbacks inherent therein.

Accordingly, an object of the present invention is to provide an athletic sock with an attached ankle brace.

Another object of the present invention is to provide an ankle supportive sock where the sock includes an attached brace that allows for adjustments in the tension provided by the brace.

To achieve the above objects, in an aspect of the present invention, a pair of ankle supportive socks is described where each sock comprises a tubular body, where the tubular body includes a foot portion, a rise and an opening; an ankle brace attached to a backside of the tubular body; a pair of wings extending from the ankle brace, where the pair of wings wrap around the tubular body; a fastening means on the pair of wings, where the fastening means secures the wings to each other around the tubular body. The ankle supportive sock may include a padded toe and padded heel to provide additional comfort and support during wear.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 depicts a perspective view of a pair of ankle supportive socks in accordance with an exemplary embodiment of the present invention; and

FIG. 2 depicts a backside view of a pair of ankle supportive socks in accordance with an exemplary embodiment of the present invention.

Like reference numerals refer to like parts throughout the description of several views of the drawings.

### DETAILED DESCRIPTION OF THE DRAWINGS

The present invention relates to a pair of socks that provide additional ankle support for a wearer, where the ankle support may be adjusted as needed during use. The invention provides a pair of ankle supportive socks for users, especially athletes, who may require additional strength and stability within their shoe. The ankle supportive socks are styled similar to conventional athletic socks, but include additional bracing around the ankle portion. With the added ankle bracing, the ankle supportive sock may protect wearers from undue ankle stress and may aid wearers with preexisting injuries.

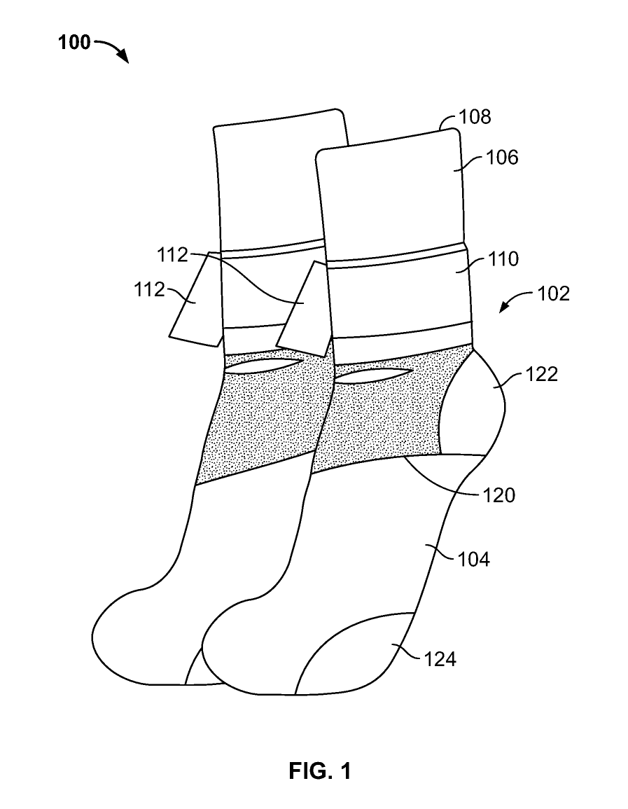

Turning now descriptively to the drawings, referring to FIG. 1, a perspective view of a pair of ankle supportive socks 100 are shown in accordance with an exemplary embodiment of the present invention. From here on the pair of socks will be described individually, but each reference is taken to apply to each sock in the pair. The ankle supportive sock 100 comprises a tubular body 102; including a foot portion 104 along a bottommost portion of the tubular body 102, and an opening 108 at a top of the tubular body 102. The tubular body 102 is shaped like a conventional athletic sock with an angled foot portion 104 and a rise 106 to the opening 108 that comes up the wearer's leg. The rise 106 may stop at the opening 108 at the mid-calf portion of the leg to provide a crew sock, or it may come all the way up to the knee to provide a knee-high sock. The sock material may be thick, durable and washable, able to wick moisture away from the foot during use. Common materials may be cotton, wool, nylon, polyester, acrylic, polypropylene, spandex, olefin or a combination thereof.

The ankle supportive sock 100 includes a padded heel 122 and a padded toe 124 for additional cushion to the wearer. The padded heel 122 and padded toe 124 include an additional lining that resists wear and tear typically experience with athletic socks. The padded heel 122 may cover the entire heel portion of the tubular body 102. The padded toe 124 may cover the entire toe portion, or may simply cover the surface under the toe or the ball of the foot, as illustrated in the figures.

Above the padded heel 122 is an ankle brace 110 that is attached on a backside of the tubular body 102. The ankle brace 110 wraps around the wearer's ankle and attaches to itself with a pair of overlapping wings 112. The wings 112 include a fastening means to adjust the tightness of the ankle brace 110. Depending on the activity performed, the user may choose to tighten the wings 112 during play, and then relax the tension when the activity is finished. Further, around the heel portion of the tubular body 102 may be a sleeve 122. The sleeve 122 may be a fitted wrap that fastens around the heel and upper arch of the foot. The sleeve 122 provides further support for the ankle brace 110 allowing the ankle supportive sock 100 to continue bracing the wearer's foot into the shoe. The ankle brace 110 and sleeve 120 may be made from a neoprene, polyester or cotton material for strength and flexibility during use.

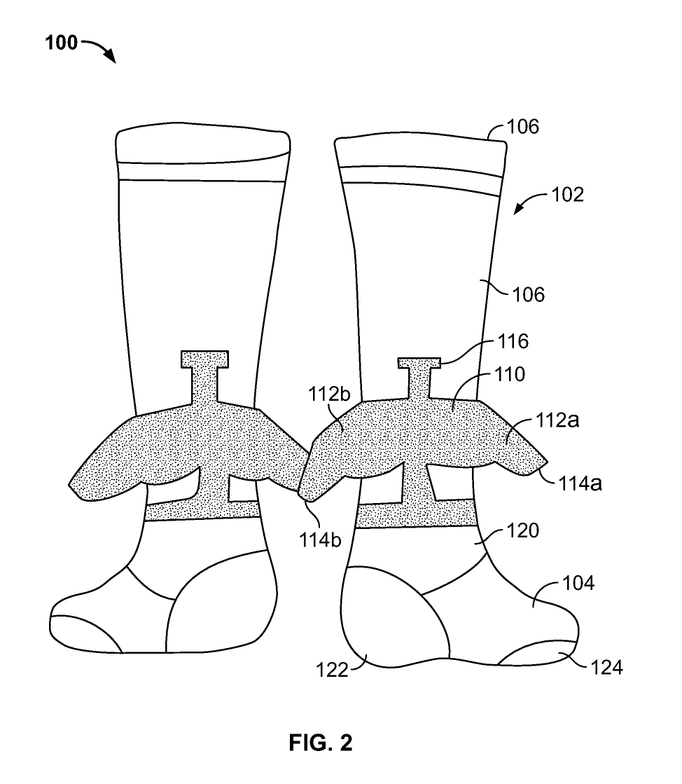

Referring now to FIG. 2, a backside view of the ankle supportive sock 100 is shown in accordance with an exemplary embodiment of the present invention. The ankle brace 110 is illustrated with the wings 112a, 112b extending laterally from the tubular body 102. The wings 112 include a fastening means 114a, 114b to secure the ankle brace 110 around the wearer's ankle. The fastening means 114a, 114b may be a hook and loop fastener, snap or laces for quick, easy and repeated adjustments while still providing a tight and secure attachment. Along a central portion of the ankle brace 110 may be a vertical support 116 which aligns with the leg of the wearer. The vertical support 116 provides additional structural reinforcement for the ankle brace which may be especially beneficial to protect the Achilles tendon.

During use, the user simply inserts their foot through the opening 106 to slide the ankle supportive sock 100 up their leg. Once their toes reach the padded toe 124 and the padded heel 122 is comfortably positioned against the heel, the user may wrap the wings 112 of the ankle brace 110 around the ankle The wings 112 are secured with the fastening means 114 to a desired tightness or tension. The wings 112 are easily detached from each other in case the ankle brace 110 is too tight or loose after initial adjustments. With the customizable function of the ankle brace 110 the wearer is afforded a comfortable yet supportive garment that they may wear whenever additional foot and ankle support is desired and necessary.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A pair of ankle supportive socks where each sock comprises:
a. a tubular body, where the tubular body includes a foot portion, a rise and an opening;
b. an ankle brace attached to a backside of the tubular body;
c. a pair of wings extending laterally from the ankle brace, where the pair of wings wrap around the tubular body; and
d. a fastening means on the pair of wings, where the fastening means secures the wings to each other around the tubular body.

2. The ankle supportive sock according to claim 1, where the foot portion includes a padded heel and a padded toe.

3. The ankle supportive sock according to claim 2, where the padded toe covers an entire toe portion of the tubular body.

4. The ankle supportive sock according to claim 2, where the padded toe covers a ball of the foot portion of the tubular body.

5. The ankle supportive sock according to claim 1, where the fastening means is one of at least a hook and loop fastener, snaps or laces.

6. The ankle supportive sock according to claim 1, where the tubular body includes a sleeve around the heel portion, where the sleeve is positioned beneath the ankle brace.

7. The ankle supportive sock according to claim 1, where ankle brace includes a vertical support.

8. The ankle supportive sock according to claim 1, where the wings of the ankle brace are adjustable to tighten and loosen the ankle brace as desired.

9. The ankle supportive sock according to claim 1, where the rise of the tubular body provides a crew sock.

10. The ankle supportive sock according to claim 1, where the rise of the tubular body provides a knee-high sock.
